(19) United States
(12) Patent Application Publication
(10) Pub. No.: US 2023/0000756 A1
(43) Pub. Date: Jan. 5, 2023

(54) COMPOSITIONS AND METHODS FOR STYLING HAIR

(71) Applicant: L'Oreal, Paris (FR)

(72) Inventors: Vibha K. SHAH, Branchburg, NJ (US); Allison PERNER, Metuchen, NJ (US); Ronak RUGHANI, Edison, NJ (US); Brady ZARKET, Union, NJ (US); Sivaramakrishnan MUTHUKRISHNAN, Bridgewater, NJ (US); Aakash PAREKH, Edison, NJ (US)

(21) Appl. No.: 17/853,128

(22) Filed: Jun. 29, 2022

Related U.S. Application Data
(60) Provisional application No. 63/217,198, filed on Jun. 30, 2021.

(30) Foreign Application Priority Data
Sep. 20, 2021 (FR) 2109871

(51) Int. Cl. A61K 8/85; A61K 8/9717; A61K 8/73; A61K 8/34; A61Q 5/06

(52) U.S. Cl. CPC A61K 8/85; A61K 8/9717; A61K 8/731; A61K 8/73; A61K 8/342; A61K 8/345; A61Q 5/06

(57) ABSTRACT

The disclosure relates to compositions comprising (a) at least one polyphenol; (b) at least one film former; (c) at least one gum; (d) at least one emulsifier; (e) at least one fatty alcohol; and (f) at least one solvent. Optionally, the compositions may further comprise at least one humectant and/or at least one fatty acid. The compositions can be used for styling hair, for example temporarily straightening the hair and/or for reducing frizz of hair. The disclosure also relates to methods of using the compositions to style hair, straighten hair, and/or reduce frizz of hair.

### TECHNICAL FIELD

The present disclosure relates to compositions and methods for styling hair, such as curly or wavy hair. The compositions provide temporary straightening properties to the hair. The disclosure also relates to methods of using the compositions to style the hair.

### BACKGROUND

Consumers with curly or wavy hair often desire to temporarily straighten their hair for a change in appearance. In addition, reducing the curls of very curly hair may increase the manageability and ease of styling of such hair.

Traditional hair-straightening products generally include heavy galenics or oils, which may leave an undesirable greasy or oily feeling. Products that provide hold, such as styling gels, can also give straightening effects, but these products typically flake on the hair. In addition, many hair straightening products involve heat or chemical treatments that may cause damage to the hair fibers and/or irritate the scalp.

As such, consumers desire new and improved compositions that can provide straightening effects to curly or wavy hair, and at the same time impart various additional advantageous properties to the hair such as softness, smoothness, shine, and/or frizz control.

It has now surprisingly been found that compositions comprising a particular combination of components as described herein are able to temporarily straighten curly or wavy hair without the use of heat or harsh chemicals, while at the same time delivering additional advantageous sensorial benefits to the hair.

### SUMMARY

The present disclosure relates to compositions for styling hair, such as curly or wavy hair. The compositions may provide beneficial effects such as straightening the hair, and/or one or more of softness, smoothness, shine, volume control, and/or frizz control to the hair. The compositions are optionally leave-in compositions, and may, in various embodiments, be in the form of spray compositions.

The hair styling compositions may comprise, in certain embodiments: (a) at least one polyphenol; (b) at least one film former; (c) at least one gum; (d) at least one emulsifier; (e) at least one fatty alcohol; and (f) at least one solvent. Optionally, the hair styling compositions may further comprise at least one humectant and/or at least one fatty acid.

The at least one polyphenol may be chosen from flavonoids and non-flavonoids, as well as mixtures thereof, and in a particular embodiment is chosen from flavonoids. For example, in various embodiments the at least one polyphenol comprises, consists essentially or, or consists of one or more flavonoid compounds. In some embodiments, the flavonoid comprises, consists essentially of, or consists of tannic acid. The at least one polyphenol may be present in an amount ranging from about 0.01% to about 10% by weight, relative to the total weight of the composition, such as, for example, about 0.1% to about 9%, about 1% to about 7%, or about 2% to about 6%.

The at least one film former may be chosen from, by way of example, carboxylic acid polymers, polysaccharides, gums, or mixtures thereof, and in some embodiments comprises, consists essentially of, or consists of carrageenan. The at least one film former may be present in an amount ranging from about 0.01% to about 5% by weight, relative to the total weight of the composition, such as, for example, about 0.1% to about 5%, about 0.1% to about 3%, or about 0.1% to about 1%.

The at least one gum may be chosen from, by way of example only, microcrystalline cellulose, cellulose gum, sclerotium gum, xanthan gum, diutan gum, or mixtures thereof, and may be present in an amount ranging from about 0.01% to about 5%, such as from about 0.1% to about 3%, about 0.2% to about 2%, or about 0.3% to about 1.5% by weight, based on the total weight of the composition.

The at least one emulsifier may be chosen from, by way of example only, crosslinked carboxylic acid polymers containing one or more monomers derived from acrylic acid, substituted acrylic acids, and salts and derivatives thereof, such as crosslinked homopolymers of an acrylic acid or of a derivative thereof, or crosslinked copolymers having (i) a first monomer selected from the group consisting of (meth)acrylic acid, derivatives thereof, short chain (e.g. C1-C4) acrylate ester monomers, or mixtures thereof, and (ii) a second monomer which is a long chain (e.g. C8-C40) substituted polyethylene glycol acrylate ester monomer. The at least one emulsifier may be present in an amount ranging from about 0.01% to about 5%, such as from about 0.1% to about 3%, about 0.5% to about 2%, or about 0.5% to about 1% by weight, based on the total weight of the composition. In some embodiments, the at least one emulsifier comprises a crosslinked anionic copolymer of acrylamide and of AMPS. In other embodiments, the emulsifier comprises Polyacrylamide, C13-14 Isoparaffin, and Laureth-7.

The at least one fatty alcohol may, in various embodiments, be chosen from straight or branched chain, saturated or unsaturated fatty alcohols having from 8 to 30 carbon atoms, from 12 to 22 carbon atoms, or from 14 to 22 carbon atoms, derivatives thereof, or mixtures thereof, such as, for example, decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, derivatives thereof, or mixtures thereof. The at least one fatty alcohol may be present in an amount ranging from about 0.01% to about 10% by weight, relative to the total weight of the composition, such as, for example, about 0.05% to about 5%, about 0.1% to about 3%, or about 0.1% to about 1%.

The solvent comprises water and optionally at least one non-aqueous solvent, and may be present in an amount ranging from about 50% to about 99%, such as about 60% to about 98%, or about 70% to about 95% by weight, relative to the total weight of the composition. In some embodiments, the solvent consists of or consists essentially of water, with the solvent comprising less than about 20%, less than about 10%, less than about 5%, or less than about 3% by weight, relative to the total weight of the composition, of non-aqueous solvents.

If present, the at least one humectant may be chosen from, for example, glycerin, propylene glycol, butylene glycol, pentylene glycol, hexylene glycol, dipropylene glycol, diethylene glycol, diglycerol, hyaluronic acid, sorbitol, xylitol, inulin, or mixtures thereof. In certain embodiments, the composition comprises at least one humectant present in an amount ranging from about 0.1% to about 10% by weight, relative to the total weight of the composition, such as, for example, from about 0.5% to about 8%, or about 1% to about 5%.

If present, the at least one fatty acid may be chosen from straight or branched chain fatty acids, which may be saturated or unsaturated, and may have, in various embodiments, from 8 to 30 carbon atoms, such as, for example, from 8 to 25, from 8 to 22, from 8 to 20, from 8 to 18, or from 8 to 16 carbon atoms, or from 12 to 30, from 12 to 25, from 12 to 22, from 12 to 20, or from 12 to 18 carbon atoms. The at least one fatty acid may be present in an amount ranging from about 0.01% to about 5% by weight, relative to the total weight of the composition, such as, for example, about 0.05% to about 4%, about 0.1% to about 3%, or about 0.5% to about 2%.

In further embodiments, the compositions further comprise at least one antioxidant, at least one preservative, at least one pH adjuster, at least one buffer, at least one vitamin, or mixtures of two or more thereof.

In certain embodiments, the hair styling compositions according to the disclosure may comprise (a) tannic acid; (b) carrageenan; (c) at least one gum chosen from microcrystalline cellulose, cellulose gum, sclerotium gum, xanthan gum, diutan gum, or mixtures thereof; (d) at least one emulsifier comprising Polyacrylamide, C13-14 Isoparaffin, and Laureth-7; (e) at least one fatty alcohol; (f) water, present in an amount of at least about 50% by weight, relative to the total weight of the composition; and (g) at least one humectant chosen from glycerin, at least one glycol, inulin, sorbitol, or mixtures thereof.

In yet further embodiments, the hair styling compositions according to the disclosure may comprise (a) from about 0.5% to about 6% of tannic acid; (b) from about 0.1% to about 1% of carrageenan; (c) from about 0.1% to about 2% of at least one gum chosen from microcrystalline cellulose, cellulose gum, sclerotium gum, xanthan gum, diutan gum, or mixtures thereof; (d) at least one emulsifier comprising Polyacrylamide, C13-14 Isoparaffin, and Laureth-7; (e) from about 1% to about 5% of at least one fatty alcohol; (f) water, present in an amount of at least about 60% by weight, relative to the total weight of the composition; and (g) at least one humectant chosen from glycerin, at least one glycol, inulin, sorbitol, or mixtures thereof.

The disclosure further relates to methods of using the compositions to style hair, such as methods of straightening hair, and/or methods of reducing frizz, wherein the methods comprise applying the composition to wet, damp, or dry hair.

For example, in various embodiments, the disclosure relates to methods of styling the hair, methods of straightening the hair, and/or methods of reducing frizz of the hair, where the methods comprise applying to the hair a composition comprising: (a) at least one polyphenol; (b) at least one film former; (c) at least one gum; (d) at least one emulsifier; (e) at least one fatty alcohol; and (f) at least one solvent comprising water. In various methods of styling, straightening, and/or reducing frizz of the hair, the at least one polyphenol is chosen from flavonoid compounds, for example comprises, consists essentially of, or consists of tannic acid, which may be present in an amount ranging from about 0.01% to about 10% by weight, relative to the total weight of the composition, such as, for example, about 0.1% to about 8%, about 0.5% to about 5%, or about 1% to about 3%. In various embodiments, the at least one film former may be chosen from, for example, carboxylic acid polymers, polysaccharides, or mixtures thereof, and in some embodiments comprises, consists essentially of, or consists of carrageenan, and may be present in an amount ranging from about 0.01% to about 5% by weight, relative to the total weight of the composition, such as, for example, about 0.1% to about 5%, about 0.1% to about 3%, or about 0.1% to about 1%. The at least one gum chosen for the compositions used in the methods of styling, straightening, and/or reducing frizz of the hair may be, for example, microcrystalline cellulose, cellulose gum, sclerotium gum, xanthan gum, diutan gum, or mixtures thereof, and may be present in an amount ranging from about 0.01% to about 5%, such as from about 0.1% to about 3%, about 0.2% to about 2%, or about 0.3% to about 1.5% by weight, based on the total weight of the composition. The at least one emulsifier may be present in an amount ranging from about 0.01% to about 5%, such as from about 0.1% to about 3%, about 0.5% to about 2%, or about 0.5% to about 1% by weight, based on the total weight of the composition, and in some embodiments comprises a crosslinked anionic copolymer of acrylamide and of AMPS; in some embodiments the emulsifier comprises Polyacrylamide, C13-14 Isoparaffin, and Laureth-7. In the methods for styling, straightening, and/or reducing frizz of hair, the at least one fatty alcohol may, in certain embodiments, be chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, derivatives thereof, or mixtures thereof, and may be present in an amount ranging from about 0.01% to about 10% by weight, relative to the total weight of the composition, such as, for example, about 0.1% to about 8%, about 0.5% to about 6%, or about 1% to about 5%. The solvent in the methods of styling, straightening, and/or reducing frizz of the hair comprises water and optionally at least one non-aqueous solvent, and may be present in an amount ranging from about 50% to about 99%, such as about 60% to about 98%, or about 70% to about 95% by weight, relative to the total weight of the composition. In some embodiments, the solvent consists of or consists essentially of water, with the solvent comprising less than about 20%, less than about 10%, less than about 5%, or less than about 3% by weight, relative to the total weight of the composition, of non-aqueous solvents.

Optionally, the compositions useful in the methods of styling, straightening, and/or reducing frizz of the hair may further comprise at least one humectant and/or at least one fatty acid. If present, the amount of the at least one humectant may range from about 0.1% to about 10% by weight, relative to the total weight of the composition, such as, for example, from about 0.5% to about 8%, or about 1% to about 5%. If present, at least one fatty acid in the compositions used in the methods for styling, straightening, and/or reducing frizz of the hair, may be chosen from straight or branched chain fatty acids, which may be saturated or unsaturated, and may have, in various embodiments, from 8 to 30 carbon atoms, such as, for example, from 8 to 25, from 8 to 22, from 8 to 20, from 8 to 18, or from 8 to 16 carbon atoms, or from 12 to 30, from 12 to 25, from 12 to 22, from 12 to 20, or from 12 to 18 carbon atoms, and may be present in an amount ranging from about 0.01% to about 5% by weight, relative to the total weight of the composition, such as, for example, about 0.05% to about 4%, about 0.1% to about 3%, or about 0.5% to about 2%.

Optionally, in order to style, straighten, and/or reduce frizz of the hair, the compositions are left on the hair for a period of a few hours to a few days, for example until the hair is washed. In various embodiments, the compositions and/or methods do not include the use of a reducing agent, and/or do not include a step of heating the hair.

The compositions are not mascara compositions, and are not intended to be applied to the eyelashes or eyebrows. Similarly, the compositions are not make-up compositions, and are not intended to be applied to the skin.

### BRIEF DESCRIPTION OF THE FIGURES

The accompanying figures, which are incorporated herein and constitute part of this specification, illustrate exemplary embodiments of the disclosure, and, together with the general description given above and the description provided herein, serve to demonstrate features of the disclosure.

FIG. 1 is a photograph showing compositions 2A and 2A′ at the end of an 8-week stability study.

FIG. 2 is a photograph showing films formed from compositions 1A and C1, after drying.

FIGS. 3A-3B are photographs of swatches of hair treated with compositions 1A, 1B, C1, and a control (water) immediately after breaking the film, and after 2 hours in a humidity chamber.

FIGS. 4A-4B are photographs of mannequin heads treated with compositions 1A, C1, and a control (water) while the hair is still wet and after the hair is dried and the film is broken.

FIG. 5 shows photographs of two volunteers' curly hair before treatment, and after treatment with compositions 1B and C1, once the hair is dried and the film is broken.

It is to be understood that the foregoing and following descriptions are exemplary and explanatory only, and are not intended to be restrictive of any subject matter claimed.

### DETAILED DESCRIPTION

The disclosure relates to compositions for styling hair, for example compositions for straightening wavy or curly hair and/or reducing frizz of the hair, as well as to methods of using the compositions.

I. Compositions

In various embodiments, compositions according to the disclosure comprise (a) at least one polyphenol; (b) at least one film former; (c) at least one gum; (d) at least one emulsifier; (e) at least one fatty alcohol; and (f) at least one solvent. Optionally, the compositions may further comprise (g) at least one humectant; and/or (h) at least one fatty acid. The compositions are optionally leave-in compositions, and may, in various embodiments, be in the form of spray compositions.

Compositions according to the disclosure may provide straightening effects to the hair, e.g. temporary straightening effects, and in at least some embodiments may provide one or more additional benefits such as softness, smoothness, shine, and/or frizz control to the hair.

### Polyphenols

Compositions according to the disclosure comprise at least one polyphenol. Polyphenols are phenols with more than one phenolic —OH group that have the ability to act as “donor molecules” by donating their alcoholic hydrogen or accepting delocalized electrons. Useful polyphenols include both flavonoids and non-flavonoids.

Exemplary and non-limiting flavonoid compounds that can be used include, for example, flavanols, such as catechin, fisetin, kaempferol, myricetin, quercetin, rutin, proanthocyanidins, pyroanthocyanidins, theaflavins, or thearubigins (or thearubrins); dihydroflavonols, such as astilbin, dihydroquercetin, or silibinin; flavanones, such as hesperidin, neohesperidin, hesperetin, naringenin, naringin, or poncirin; flavones, such as apigenin, baicalin, diosmin, or rhoifolin; tannins, such as ellagitannins, tannic acid, gallic acid, or ellagic acid; isoflavonoids, such as biochanin A, Daidzein, or Genistein; fulvic acid, and neoflavonoids, as well as combinations thereof.

Exemplary and non-limiting non-flavonoid compounds that can be used include, for example, stibenoids such as astringin, resveratrol, or rhaponticin, as well as combinations thereof.

Other polyphenols that can be used include hydroxycinnamic acids, for example, chlorogenic acid, verbascoside; phenolic aldehydes; phenylpropenes; coumarins, coumestans, or tyrosols, as well as combinations thereof. In one embodiment, the polyphenols may be plant-based and/or organic.

In certain exemplary embodiments, polyphenols useful according to the disclosure may be chosen from tannic acid, resveratrol, catechin, ellagic acid, resorcinol, gallic acid, humic acid, chlorogenic acid, quercetin, chebulinic acid, or mixtures thereof. In certain embodiments, the polyphenols are chosen from polyphenols that do not impart color to the hair. In some embodiments, the polyphenol comprises, consists essentially of, or consists of tannic acid.

In various embodiments, the at least one polyphenol is present in the compositions in an amount of about 0.01% to about 10% by weight, such as about 0.1% to about 8%, about 0.5% to about 5%, or about 1% to about 3% by weight, relative to the total weight of the composition. By way of non-limiting example, the at least one polyphenol is present in the composition with a total amount of polyphenols ranging from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, from about 0.75% to about 3%, from about 1% to about 10%, from about 1% to about 9%, from about 1% to about 8%, from about 1% to about 7%, from about 1% to about 6%, from about 1% to about 5%, from about 1% to about 4%, or from about 1% to about 3% by weight, relative to the total weight of the composition, including all ranges and subranges thereof.

In some embodiments, the at least one polyphenol comprises, consists essentially of, or consists of one or more flavonoid compounds, for example tannic acid. In some embodiments, the composition comprises a total amount of flavonoid compounds ranging from about 0.1% to about 5%, such as about 0.1% to about 4%, about 0.1% to about 3%, about 1% to about 5%, about 1% to about 4%, or about 1% to about 3% by weight, relative to the total weight of the composition, including all ranges and subranges thereof. For example, the composition may comprise tannic acid, present in an amount ranging from about 0.1% to about 5%, such as about 0.1% to about 4%, about 0.1% to about 3%, about 1% to about 5%, about 1% to about 4%, or about 1% to about 3% by weight, relative to the total weight of the composition, including all ranges and subranges thereof.

### Film Formers

Compositions according to the disclosure comprise at least one film forming agent, referred to interchangeably herein as “film former.”

In various embodiments, the at least one film former may be chosen from, by way of example only, carboxylic acid polymers, polysaccharides, or mixtures thereof.

By way of non-limiting example, the at least one film former may be chosen from carbomers, acrylates/C10-C30 alkyl acrylate crosspolymers, carrageenan, calcium carrageenan, potassium carrageenan, sodium carrageenan, or mixtures thereof. In various embodiments, the at least one film former is chosen from neutral acrylic homopolymers and copolymers, C1-C2 alkyl celluloses, C1-C3 alkyl guar, C1-C3 hydroxyalkyl guar, or mixtures thereof. In some embodiments, the film former does not comprise any homopolymers or copolymers of ethylene oxide, polyvinyl alcohols, homopolymers or copolymers of vinylpyrrolidone, homopolymers or copolymers of vinylcaprolactam, and/or homopolymers or copolymers of polyvinyl methyl ether. In further embodiments, the film former does not comprise a nonionic polymer other than those expressly disclosed herein.

The at least one film former is present in the composition in an amount ranging from about 0.01% to about 5%, such as from about 0.01% to about 4%, about 0.01% to about 3%, about 0.01% to about 2%, about 0.01% to about 1%, about 0.01% to about 0.8%, about 0.05% to about 5%, about 0.05% to about 4%, about 0.05% to about 3%, about 0.05% to about 2%, about 0.05% to about 1%, about 0.05% to about 0.8%, about 0.1% to about 5%, about 0.1% to about 4%, about 0.1% to about 3%, about 0.1% to about 2%, about 0.1% to about 1%, about 0.1% to about 0.9%, about 0.1% to about 0.8%, about 0.1% to about 0.7%, or about 0.1% to about 0.6% by weight, including all ranges and sub-ranges there between, based on the total weight of the composition.

In one exemplary embodiment, the at least one film former comprises, consists essentially of, or consists of carrageenan, and is present in the composition in an amount ranging from about 0.1% to about 5%, about 0.1% to about 4%, about 0.1% to about 3%, about 0.1% to about 2%, about 0.1% to about 1%, about 0.1% to about 0.9%, about 0.1% to about 0.8%, about 0.1% to about 0.7%, or about 0.1% to about 0.6% by weight, including all ranges and sub-ranges there between, based on the total weight of the composition.

### Gums

Compositions according to the disclosure comprise at least one gum. According to the disclosure, the at least one gum is different than, and in addition to, the at least one film former.

Non-limiting examples of gums that may be chosen for the at least one gum include acacia, agar, algin, alginic acid, ammonium alginate, amylopectin, calcium alginate, carboxymethyl hydroxyethylcellulose, carnitine, cellulose acetate propionate carboxylate, cellulose gum, dextrin, diutan gum, gelatin, gellan gum, guar gum, hydroxyethylcellulose, hydroxypropylcellulose, hydroxypropyl methylcellulose, hydroxypropyl chitosan, hydroxypropyl guar, karaya gum, kelp, locust bean gum, methyl hydroxyethylcellulose, microcrystalline cellulose, natto gum, pectin, potassium alginate, propylene glycol alginate, sclerotium gum, sodium carboxymethyl dextran, sodium cellulose sulfate, tragacanth gum, xanthan gum, biosaccharide gum, and mixtures thereof. In certain embodiments, the at least one gum comprises, consists essentially of, or consists of microcrystalline cellulose, cellulose gum, sclerotium gum, xanthan gum, diutan gum, or mixtures thereof.

The at least one gum may be present in an amount ranging from about 0.01% to about 5%, such as from about 0.1% to about 3%, about 0.2% to about 2%, about 0.3% to about 1.5%, or about 0.3% to about 1%, based on the total weight of the composition, including all ranges and subranges thereof. For example, the at least one gum may range from about 0.01% to about 5%, about 0.01% to about 4%, about 0.01% to about 3%, about 0.01% to about 2%, about 0.01% to about 1%, about 0.01% to about 0.5%, about 0.05% to about 5%, about 0.05% to about 4%, about 0.05% to about 3%, about 0.05% to about 2%, about 0.05% to about 1%, about 0.05% to about 0.5%, about 0.1% to about 5%, about 0.1% to about 4%, about 0.1% to about 3%, about 0.1% to about 2%, about 0.1% to about 1%, about 0.1% to about 0.5%, or about 0.1% to about 0.3% by weight, based on the total weight of the composition, including all ranges and subranges thereof. In certain embodiments, the at least one gum may be present in an amount of ranging from about 0.1% to about 1%, such as about 0.1% to about 0.8%, about 0.1% to about 0.7%, about 0.1% to about 0.6%, about 0.1% to about 0.5%, about 0.1% to about 0.4%, or about 0.1% to about 0.3% by weight, including all ranges and sub-ranges there between, based on the total weight of the composition.

In some embodiments, the at least one gum is present in an amount ranging from about 0.2% to about 0.8%, about 0.3% to about 0.7%, or about 0.4% to about 0.6% by weight, based on the total weight of the composition, including all ranges and subranges thereof. In various embodiments, the at least one gum comprises, consists essentially of, or consists of microcrystalline cellulose, cellulose gum, sclerotium gum, xanthan gum, diutan gum, or mixtures thereof, and is present in an amount ranging from about 0.1% to about 1%, such as about 0.4% to about 0.6% by weight, based on the total weight of the composition.

In certain embodiments, it may be desirable to include at least two gums, wherein the amounts of first gum and second gum are present in the compositions in a particular weight ratio for advantageous benefits. For example, the weight ratio of first gum:second gum may range from about 1:5 to about 1:15, such as from about 1:6 to about 1:14, from about 1:7 to about 1:13, from about 1:8 to about 1:12, from about 1:9 to about 1:11, or about 1:10, including all ranges and subranges thereof. By way of example only, the at least one first gum may be chosen from xanthan gum and the at least one second gum may be chosen from microcrystalline cellulose gum, and the weight ratio of xanthan gum:microcrystalline cellulose gum may range from about 1:5 to about 1:15, such as from about 1:6 to about 1:14, from about 1:7 to about 1:13, from about 1:8 to about 1:12, from about 1:9 to about 1:11, or about 1:10.

### Emulsifiers

Compositions according to the disclosure comprise at least one emulsifier, preferably a polymeric emulsifier.

Non-limiting examples of emulsifiers that can be chosen include carboxylic acid polymers which are crosslinked compounds containing one or more monomers derived from acrylic acid, substituted acrylic acids, and salts and derivatives of these acrylic acids and substituted acrylic acids, such as crosslinked homopolymers of an acrylic acid or of a derivative thereof, e.g. acrylamidopropylsulfonic acid, or crosslinked copolymers having (i) a first monomer selected from the group consisting of (meth)acrylic acid, derivatives thereof, short chain (e.g. C1-C4) acrylate ester monomers, and mixtures thereof, and (ii) a second monomer which is a long chain (e.g. C8-C40) substituted polyethylene glycol acrylate ester monomer. Other non-limiting examples of emulsifiers include ethylene oxide/propylene oxide block copolymers; alkylpolyglucosides; phospholipids; ethoxylated esters of derivatives of natural oils such as the polyethoxylated ester of hydrogenated castor oil available commercially for example under the trade name Arlacel 989 (ICI); anionic emulsifiers such as fatty acid soaps e.g., potassium stearate and fatty acid sulphates e.g., sodium cetostearyl sulphate available commercially under the trade name Dehydag (Henkel); ethoxylated fatty alcohols, for example the emulsifiers available commercially under the trade name Brij (ICI); methylglucose esters such as polyglycerol-3 methyl glucose distearate available commercially under the name Tegocare 450 (Degussa Goldschmidt); polyacrylamide emulsifier systems, or mixtures thereof.

The at least one emulsifier may be present in the composition in an amount ranging from about 0.01% to about 5%, such as from about 0.1% to about 3%, about 0.2% to about 2%, about 0.3% to about 1%, or about 0.3% to about 0.8% by weight, based on the total weight of the composition, including all ranges and subranges thereof. For example, the at least one emulsifier may range from about 0.01% to about 5%, about 0.01% to about 4%, about 0.01% to about 3%, about 0.01% to about 2%, about 0.01% to about 1%, about 0.05% to about 5%, about 0.05% to about 4%, about 0.05% to about 3%, about 0.05% to about 2%, about 0.05% to about 1%, about 0.1% to about 5%, about 0.1% to about 4%, about 0.1% to about 3%, about 0.1% to about 2%, about 0.1% to about 1%, about 0.5% to about 5%, about 0.5% to about 4%, about 0.5% to about 3%, about 0.5% to about 2%, or about 0.5% to about 1% by weight, based on the total weight of the composition, including all ranges and subranges thereof.

In one embodiment, the at least one emulsifier is a polymeric emulsifier and is present in an amount ranging from about 0.01% to about 10%, about 0.01% to about 5%, about 0.1% to about 5%, about 0.1% to about 3%, about 0.1% to about 2%, or about 0.1% to about 1%, about 0.5% to about 5%, about 0.5% to about 3%, about 0.5% to about 2%, or about 0.5% to about 1% by weight, based on the total weight of the composition, including all ranges and subranges thereof.

In one embodiment, the at least one polymeric emulsifier comprises a crosslinked anionic copolymer of acrylamide and of AMPS, e.g. in the form of a water-in-oil emulsion, such as those sold under the name SEPIGEL 305 (Polyacrylamide and C13-14 Isoparaffin and Laureth-7) or under the name SIMULGEL 600 (Acrylamide/Sodium acryloyldimethyltaurate copolymer and isohexadecane and Polysorbate 80) by SEPPIC. In one embodiment, the emulsifier comprises Polyacrylamide, C13-14 Isoparaffin, and Laureth-7.

### Fatty Alcohols

Compositions according to the disclosure comprise at least one fatty alcohol or fatty alcohol derivative.

Useful and non-limiting examples of fatty alcohols include those having from about 8 to about 30 carbon atoms, from about 12 to about 22 carbon atoms, or from about 14 to about 22 carbon atoms. The fatty alcohols can be straight or branched chain alcohols, and can be saturated or unsaturated. For example, the fatty alcohols may be chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, derivatives thereof, or mixtures thereof. In some cases, the fatty alcohols comprise at least one of, or may be chosen from, myristyl alcohol, cetyl alcohol, stearyl alcohol, cetearyl alcohol, isostearyl alcohol, oleyl alcohol, isotridecyl alcohol, derivatives thereof, or mixtures thereof.

The at least one fatty alcohol may be present in the composition in an amount ranging from about 0.01% to about 10% by weight, such as about 0.1% to about 5%, about 0.1% to about 3%, or about 0.1% to about 1% by weight, relative to the total weight of the composition. By way of non-limiting example, the at least one fatty alcohol is present in the composition with a total amount ranging from about 0.01% to about 5%, about 0.01% to about 4%, about 0.01% to about 3%, about 0.01% to about 2%, about 0.01% to about 1%, about 0.05% to about 5%, about 0.05% to about 4%, about 0.05% to about 3%, about 0.05% to about 2%, about 0.05% to about 1%, about 0.1% to about 5%, about 0.1% to about 4%, about 0.1% to about 3%, about 0.1% to about 2%, about 0.1% to about 1%, or about 0.1% to about 0.5% by weight, relative to the total weight of the composition, including all ranges and subranges thereof.

### Solvent

Compositions according to the disclosure comprise one or more cosmetically acceptable solvents. The solvent(s) may be chosen from water, non-aqueous solvents, or mixtures thereof. The total amount of solvent in the composition may range from about 50% to about 99% by weight, relative to the total weight of the composition, including all ranges and subranges therebetween. For example, in one embodiment, the total amount of solvent may be about 50% to about 98%, about 60% to about 95%, about 70% to about 90%, about 70% to about 85%, or about 75% to about 85% by weight, relative to the total weight of the composition.

In one embodiment, the solvent comprises water. In other embodiments, the composition comprises water and one or more non-aqueous solvents. For example, C₁-C₄ alcohols, organic solvents, polyols, glycols, hydrocarbons/alkanes, glycol ethers, or any mixture thereof. Non-limiting examples of solvents which may be used include alkane polyols such as, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-isopropyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-isopropyl ether, dipropylene glycol monomethyl ether, dipropylene glycol monoethyl ether, dipropylene glycol mono-n-propyl ether, and dipropylene glycol mono-isopropyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In some exemplary embodiments, the solvent comprises water and at least one additional solvent chosen from linear or branched, saturated or unsaturated, and substituted or unsubstituted polyols, for example C₂-C₈ polyols or C₃-C₆ polyols, such as diols and/or triols. Any stereoisomer of the polyols may be used. In various exemplary embodiments, the C₂-C₈ polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, and mixtures thereof.

In various embodiments, the solvent consists of, or consists essentially of, water. In certain embodiments, the solvent comprises water in an amount ranging from about 50% to about 95%, about 60% to about 90%, about 70% to about 85%, or about 75% to about 80%, by weight of the solvent, with the remainder of the solvent comprising one or more additional solvents. For example, in certain embodiments, the composition comprises about 50% to about 99%, about 60% to about 98%, about 70% to about 95%, or about 75% to about 93% water, by weight of the composition. In other embodiments, the compositions comprise water and at least one non-aqueous solvent, where the total amount of non-aqueous solvent is present in an amount less than about 20%, such as less than about 18%, less than about 16%, less than about 15%, less than about 13%, less than about 12%, less than about 10%, less than 8%, less than 5%, or less than 3% by weight, relative to the total weight of the composition. In some embodiments, the compositions are free or substantially free of non-aqueous solvents.

### Humectants

Compositions according to the disclosure optionally comprise at least one humectant. In certain embodiments, compositions according to the disclosure comprise at least two humectants.

Non-limiting humectant compounds that can be chosen include, for example, polyhydric alcohols, such as C2-C8 or C3-C6 polyhydric alcohols. By way of example, glycerol (glycerin), propylene glycol, butylene glycol, pentylene glycol, hexylene glycol, dipropylene glycols, diethylene glycol, and diglycerol may be chosen.

Additional non-limiting examples of humectants that can be chosen include hyaluronic acid, sugars such as sorbitol or xylitol, or beta-fructans, for example inulin. Mixtures of two or more humectants can also be chosen.

The humectants can be present in the composition in a total amount ranging from about 0.1% to about 10%, such as from about 0.5% to about 8%, or about 1% to about 5% by weight, based on the total weight of the composition, including all ranges and subranges thereof.

### Fatty Acids

Optionally, the compositions may comprise at least one fatty acid. For example, useful fatty acids include straight or branched chain fatty acids, which may be saturated or unsaturated. The fatty acids may, in some embodiments, have from 8 to 30 carbons, such as, for example, from 8 to 25, from 8 to 22, from 8 to 20, from 8 to 18, or from 8 to 16 carbons. In some cases, the fatty acid has from 12 to 30, from 12 to 25, from 12 to 22, from 12 to 20, or from 12 to 18 carbons. In further cases, the fatty acids may comprise C8-C22 or C14-C22 fatty groups, which may be saturated or unsaturated linear alkyl chains. The fatty acids may optionally have fatty groups chosen from stearate, isostearate, linoleate, oleate, behenate, arachidonate, palmitate, myristate, laurate, caprate, isostearyl, stearyl, linoleyl, oleyl, behenyl, myristyl, lauryl, or capryl chains, or mixtures thereof.

Non-limiting examples of fatty acids include diacids, triacids, and other multiple acids, as well as salts of these fatty acids. For example, the fatty acid may optionally include or be chosen from lauric acid, myristic acid, palmitic acid, stearic acid, behenic acid, archidonic acid, oleic acid, isostearic acid, sebacic acid, ricinoleic acid, 12-hydroxy stearic acid, or mixtures thereof. The fatty acids may, in some embodiments, be selected from the group consisting of palmitic acid, myristic acid, stearic acid, or mixtures thereof.

If present, the at least one fatty acid may be present in the composition in an amount ranging from about 0.01% to about 5% by weight, such as about 0.05% to about 4%, about 0.1% to about 3%, or about 0.5% to about 2% by weight, relative to the total weight of the composition. By way of non-limiting example, the at least one fatty acid is present in the composition with a total amount ranging from about 0.01% to about 5%, about 0.01% to about 4%, about 0.01% to about 3%, about 0.01% to about 2%, about 0.01% to about 1%, about 0.05% to about 5%, about 0.05% to about 4%, about 0.05% to about 3%, about 0.05% to about 2%, about 0.05% to about 1%, about 0.1% to about 5%, about 0.1% to about 4%, about 0.1% to about 3%, about 0.1% to about 2%, about 0.1% to about 1%, about 0.5% to about 5%, about 0.5% to about 4%, about 0.5% to about 3%, about 0.5% to about 2%, or about 0.5% to about 1% by weight, relative to the total weight of the composition, including all ranges and subranges thereof.

### Additional Components

Compositions according to the disclosure may optionally comprise any additional or auxiliary component suitable for use in such compositions. Such components may include, but are not limited to, conditioning agents, moisturizing agents, fillers, structuring agents, shine agents, antioxidants (e.g. tetradibutyl pentaerithrityl hydroxyhydrocinnamate, butylated hydroxyanisole, and/or butylated hydroxytoluene), penetrants, sequestrants, fragrances, buffers (e.g. citric acid and/or sodium citrate), dispersants, plant extracts, preserving agents (preservatives), opacifiers, sunscreen agents, pH adjusting agents, vitamins (e.g. tocopherol, ascorbic acid, and/or ascorbyl glucoside), and antistatic agents.

Optional additional or auxiliary components may be present in an amount ranging up to about 15%, such as from about 0.01% to about 15%, about 0.1% to about 10%, or about 0.1% to about 5% by weight, relative to the total weight of the composition.

In various embodiments, the compositions may have a pH less than or equal to about 7, such as from about 3 to about 6.8, from about 3.5 to about 6.5, from about 4 to about 6.5, from about 4.5 to about 6.5, from about 5 to about 6.5 or from about 5.5 to about 6.5. In other embodiments, the compositions may have a pH less than or equal to about 6, such as from about 3 to about 6, from about 3 to about 5, or from about 3 to about 4.5. In some embodiments, the pH of the compositions may be substantially unchanged (e.g. may have a change in pH of less than 20%, less than 15%, or less than 10%) when stored under ambient conditions for 4 weeks or more, such as 8 weeks.

Compositions according to the disclosure are typically in the form of a liquid, for example a liquid that is suitable for application as a spray-on composition, or may be a sprayable gel. The composition may be an aerosol, in which case a propellant may be used, or may be in non-aerosol form, such as a pump-type spray.

II. Methods

The present disclosure also relates to methods for styling hair, for example by temporarily straightening curly or wavy hair by application of the compositions described herein to the hair. The methods generally comprise applying any of the compositions according to the disclosure to the hair.

The compositions may be useful in a variety of settings, either for chemically treated or untreated hair, and/or for natural or unnatural curls. The degree of curliness or curl type of the hair may vary and is not limited. For example, the curls of hair been treated by the compositions disclosed herein may range from slightly wavy to very kinky and coily hair, and may have different textures and colors.

In addition to temporarily straightening the hair, the compositions are surprisingly capable of providing a variety of desirable sensory benefits, for example, moisture, smoothness, softness, shine, and/or anti-frizz properties, to the hair. As such, the compositions are useful in styling (which includes straightening) hair while also caring for hair, conditioning hair, and/or imparting one or more above-described sensory benefits to the hair.

Typically, methods according to the disclosure may include applying an effective amount of a composition disclosed herein to hair having waves or curls, when the hair is dry, wet, damp, or moist. The hair may be either natural or synthetic hair. As used herein, the term “effective amount” refers to an amount sufficient to provide a desired straightening effect to the hair, taking into account the degree of curliness, the length, the volume, and the texture of the hair, and/or an amount sufficient to provide a desired sensory benefit to the hair. In general, from about 0.5 grams to about 50 grams of product is applied to the hair, depending on the specific product formulation, hair length, hair volume, and hair style type. The composition applied to the hair may, for example, be distributed evenly by combing through with fingers or a means such as a comb or the like. The composition may then be dried, for example air-dried or dried with a hair dryer. Heat may optionally be used during drying, but is not required. The composition may be allowed to remain on the hair as a leave-in product for any period of time as needed, for example, a few hours or a few days, or until the next washing or rinsing of the hair. Alternatively, the compositions may be rinsed from the hair after a brief leave-in period, from about a few seconds (e.g. 5, 10, 20, 30, or 60 seconds) to about 10, 20, or 30 minutes, or longer.

In some embodiments, before applying the composition to the hair, the hair may be first cleansed with a commercially available shampoo, and/or rinsed with water, and/or dried, either partially or completely. The composition may then be applied to the washed and/or rinsed hair when the hair is wet, damp, moist, or dry. In some other embodiments, before applying the composition to dry hair, the hair can optionally be moistened, damped, or wetted by water spray or using a wet towel, or by applying other treatment compositions that make the hair moist, damp, or wet.

It has surprisingly and unexpectedly been discovered that methods according to the disclosure generally do not require the use of a reducing agent, including a base, or heating the hair in order to achieve a temporary straightening effect. As such, in some embodiments, the method does not include using a reducing agent or a base, and/or does not including heating the hair.

In various embodiments, the methods of treating hair with the compositions according to the disclosure impart the benefits described above to the hair, without a greasy feel or flaking, relative to hair not having been treated with a composition according to the disclosure. The benefits imparted to the hair may remain a desired length of time, such as a few hours, a few days, or until the hair is rinsed or washed.

The compositions described herein are not mascara compositions, i.e. are not intended to be applied to eyelashes and/or eyebrows. The compositions are likewise not makeup compositions, i.e. are not intended to be applied to skin, in particular, to skin on the face, body, or lips. Thus, treatment of, or application to, the hair is intended to be limited to hair on the head, i.e. the scalp of the head, and not eyelashes, eyebrows, body or facial hair, or the like. In such embodiments, a skilled person understands that “hair on the head” means hair that grows from the scalp of the head, and excludes eyebrows, eyelashes, facial hair, etc.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, or F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, and a salt of F may be included. However, it is to be understood that such language also includes embodiments where G, H, I, etc., are present, as long as at least one of A, B, C, D, E, F, or a salt thereof, is also present.

The term “and/or” should be understood to include both the conjunctive and the disjunctive. For example, “A and/or B” means “A and B” as well as “A or B,” and expressly covers instances of either, without reference to the other. For example, a composition that comprises “at least one humectant and/or at least one fatty acid” means that the disclosure includes compositions comprising one or more humectants or one or more fatty acids, as well as compositions comprising one or more humectants and one or more fatty acids. As a further example, disclosure of “methods of styling the hair, straightening the hair, and/or reducing frizz of the hair” includes methods of styling the hair or methods of straightening the hair or methods of reducing frizz of the hair, as well as methods of styling the hair and straightening the hair, methods of styling the hair and reducing frizz of the hair, methods of styling the hair and straightening the hair and reducing frizz of the hair, etc.

As used herein, the phrases “and mixtures thereof,” “and a mixture thereof,” “and combinations thereof,” “and a combination thereof,” “or mixtures thereof,” “or a mixture thereof,” “or combinations thereof,” “or a combination thereof,” and variations thereof are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Further, unless expressly stated otherwise or when the context of the disclosure clearly dictates otherwise, the language “the at least one X may be present in an amount ranging from Y % to Z %” is to be understood to mean that the total amount of X's present in the composition ranges from Y % to Z %, inclusive of the endpoints. For example, “the at least one polyphenol may be present in an amount ranging from about 0.01% to about 10%” means that the total amount of polyphenols present ranges from about 0.01% to about 10%, inclusive of both 0.01% and 10%.

“Active material” as used herein with respect to the percent amount of an ingredient or raw material, refers to 100% activity of the ingredient or raw material.

All amounts given herein are relative to the amount of active material, unless otherwise indicated.

Unless otherwise indicated, all percentages herein are by weight, relative to the total weight of the composition.

All percentages, parts and ratios herein are based upon the total weight of the compositions of the present disclosure, unless otherwise indicated.

As used herein, the term “treat” (and its grammatical variations, such as “treating”) refers to the application of the compositions of the present disclosure onto the surface of hair on the head.

As used herein, the phrase “applying a composition onto hair,” and variations thereof is intended to mean contacting the hair with at least one of the compositions of the disclosure, in any manner. It may also mean contacting the hair in an effective amount.

As used herein, the term “styling” is intended to include “shaping” and “straightening.”

As used herein, the term “curly hair” refers to any hair including a curl, and “wavy hair” refers to any hair including a wave. The curl and/or wave may be natural or unnatural, i.e., formed by chemical treatment or physical treatment of the hair. The degree of curliness and/or waviness of the hair may vary and is not limited.

A “leave-in” composition or product refers to a hair-treatment composition that is not rinsed and/or washed away with water or acceptable solvent after the application of the composition onto the hair; instead, the composition is allowed to remain on the hair for a period of time as desired, such from 1 hour, 2 hours, 3 hours, 4 hours, up to 8 hours, overnight, or as long as needed, until next time of washing or rinsing the hair.

As used herein, the term “polyol” refers to an organic molecule comprising at least two free hydroxyl groups.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, unless expressly stated otherwise, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “salts” refers to throughout the disclosure may include salts having a counter-ion, in either ionized or un-ionized form. It is to be understood that, with regard to salts of acids described herein, it is intended to encompass the use of a salt of the acid as an ingredient added to a composition according to the disclosure, or to the salt of the acid that forms when the acid is used as an ingredient in a composition according to the disclosure (in ionized or un-ionized form).

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “temporary” should be understood to indicate that some degree of benefit or effect is imparted to the hair that remains from when the hair is treated with a composition according to the disclosure, until the composition is removed from the hair, e.g. by washing the hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition, unless otherwise noted.

### Example 1—Compositions

The inventive and comparative compositions set forth in Table 1 below were prepared.

TABLE 1

| | Inventive | | Comparative |
|---|---|---|---|
| | 1A | 1B | C1 |
| GLYCERYL STEARATE SE | 0.30 | 0.15 | |
| XANTHAN GUM | 0.10 | 0.10 | |
| TANNIC ACID | 5.0 | 5.0 | 5.0 |
| GLYCERIN | 3.0 | 3.0 | |
| CETEARYL ALCOHOL | 0.30 | 0.15 | |
| ISOPROPYL ISOSTEARATE | 2.0 | 2.0 | |
| CARRAGEENAN | 0.50 | 0.50 | 0.50 |
| INULIN | 1.94 | 1.94 | |
| CELLULOSE GUM | 0.15 | 0.15 | |
| MICROCRYSTALLINE | 0.85 | 0.85 | |
| CELLULOSE | | | |
| LAURETH-7/POLY- | 0.68 | 0.68 | |
| ACRYLAMIDE/C13-14 | | | |
| ISOPARAFFIN | | | |
| ADDITIVES (Vitamins, | 0.82 | 0.82 | |
| preserving agents, pH | | | |
| adjusters) | | | |
| WATER | QS to 100 | QS to 100 | QS to 100 |

The compositions were prepared as follows. First, the solvent phase was heated to a temperature of about 70-75° C. The gum, polymer, and polyphenol components were added individually with mixing at medium speed using a rotor-stator blade on a Rayneri V2004 mixer. Once the polyphenol, polymers, and gum were fully dispersed, the rest of the ingredients were added one by one with mixing at medium speed to ensure each was fully incorporated, and an emulsion was formed. Then the batch was cooled to a temperature of about 30-35° C., while mixing with a propeller-type blade.

Each of compositions 1A-1B and C1 provided temporary straightening benefits to hair, and reduced frizz even in high humidity. However, hair treated with composition C1 felt more stiff and coated than hair treated with compositions 1A-1B, which had a smoother, more natural feel.

### Example 2A—Stability Testing

The stability of the following inventive and comparative compositions was evaluated by measuring the pH and visual appearance of the compositions, over time. The inventive and comparative compositions set forth in Table 2A below were prepared following the procedure described in Example 1.

TABLE 2A

| | Inventive | | Comparative | | |
|---|---|---|---|---|---|
| | 2A | 2B | C2 | C3 | C4 |
| TANNIC ACID | 5.0 | 5.0 | 5.0 | 5.0 | 5.0 |
| CARRAGEENAN | 0.50 | 0.50 | 0.50 | 0.50 | 0.50 |
| XANTHAN GUM | 0.10 | 0.10 | | 0.10 | |
| GLYCERIN | 3.0 | 3.0 | | | |
| INULIN | 2.0 | 2.0 | | | |
| GLYCERYL STEARATE SE + | 2.6 | 2.3 | | | |
| CETEARYL ALCOHOL + | | | | | |
| ISOPROPYL ISOSTEARATE | | | | | |
| MICROCRYSTALLINE | 1.0 | 1.0 | | 1.0 | |
| CELLULOSE | | | | | |
| LAURETH-7/POLY- | 1.0 | 1.0 | | | 2.0 |
| ACRYLAMIDE/C13-14 | | | | | |
| ISOPARAFFIN | | | | | |
| PRESERVATIVES | 0.9 | 0.9 | 0.9 | 0.9 | 0.9 |
| WATER | QS to 100 | QS to 100 | QS to 100 | QS to 100 | QS to 100 |

Each of compositions 2A-2B3 and 02-04 was evaluated for stability for 10 days at 6000, and for 2 months at 4500. The results of the stability study are set forth below in Table 2B.

TABLE 2B

| | Inventive | | Comparative | | |
|---|---|---|---|---|---|
| | 2A | 2B | C2 | C3 | C4 |
| Visual | Color shift: | Color shift: | Color shift: | Color shift: | Color shift: |
| appearance | beige to dark | beige to dark | light beige to | light beige to | light beige to |
| | beige | beige | dark brown; | dark brown; | dark brown |
| | | | some | some phase | |
| | | | precipitation | separation | |
| | | | noted | noted | |
| ΔpH | ~0.5 units | ~0.5 units | ~1.2 units | ~1.0 units | ~0.6 units |

As Table 2B shows, inventive compositions 2A-21B demonstrated better stability than comparative compositions 02-04. The inventive compositions demonstrated less change in visual appearance and pH over the time period of study. This study shows that the combination of components according to the disclosure provides the greatest degree of stability of the compositions.

### Example 2B—Stability Testing

In order to prepare a composition according to the disclosure with even greater stability, 0.07% tetradibutyl pentaerithrityl hydroxyhydrocinnamate was added to composition 2A (designated composition 2A′). The stability of compositions 2A and 2A′ was then evaluated over a period of 8 weeks at 45° C.

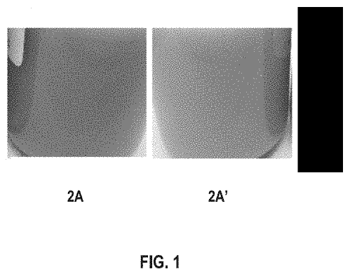

At the end of the 8-week study, a slight difference in color of compositions 2A and 2A′ was observed. Specifically, while both compositions 2A and 2A′ had started the study at a beige color, at the end of the study composition 2A was slightly darker in color than composition 2A′, demonstrating that the addition of the antioxidant had provided greater stability. FIG. 1 is a photograph showing compositions 2A and 2A′ at the end of the 8 week study, where the difference in color is visually observable.

### Example 3—Film Quality

Films produced by compositions 1A and C1 were evaluated to determine film quality. Equal amounts of compositions 1A and C1 were weighed into identical weigh boats made of polyethylene terephthalate (PET), and allowed to completely dry in a dessicator.

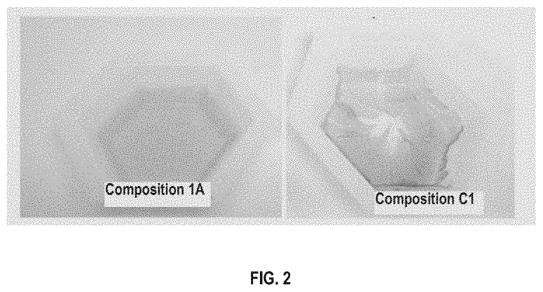

Once dry, the film formed by composition C1 was brittle and stiff, whereas the film formed by composition 1A was continuous and not as brittle and stiff as the film of composition C1, and was observed to adhere to the weigh boat well. The dried films are shown in FIG. 2.

### Example 4A—Testing on Hair

Compositions 1A, 1B, and C1, as well as water (control), were tested on swatches of curly hair. First, the hair was washed with a standard shampoo, and excess water was squeezed from the swatches by hand. Next, one of compositions 1A, 1B, C1, or the control was applied to a swatch in an amount of 0.15 grams per gram of hair, and distributed through the hair by hand (10 passes) and comb (3 passes). The swatches were then air dried under ambient conditions.

Once the hair was dry, the hair was combed with the fingers (10 passes) to break the film that had formed on the hair after drying, and the swatches were placed in a humidity chamber at room temperature and 80% relative humidity. The hair was evaluated visually.

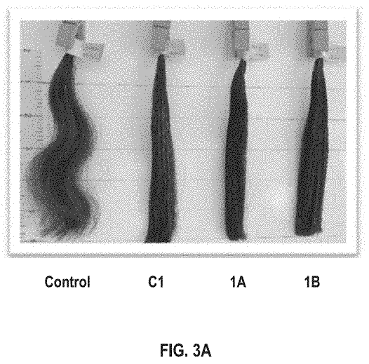

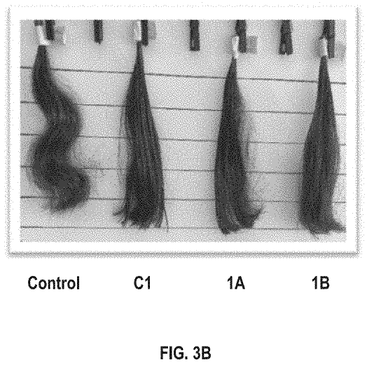

Immediately after breaking the film (FIG. 3A) and after 2 hours in the humidity chamber (FIG. 3B), the hair treated with compositions 1A, 1B, or C1 demonstrates significant straightening benefits compared to hair treated with the control. The hair treated with compositions 1A and 1B had better sensorial properties than the hair treated with composition C1, however, in particular it felt smoother and more natural. In addition, the hair treated with composition 1A felt smoother and more coated than either compositions 1B or C1.

### Example 4B—Testing on Hair

Compositions 2A and C2 were tested on a mannequin head with curly hair. First, the hair was washed and conditioned with a standard shampoo and conditioner, and then excess water was removed with a towel. Next, equal amounts of composition 2A or composition C2 was applied to one-half of the mannequin head in an amount of 5 grams per half-head, and distributed through the hair by hand, after which the hair was dried with a rollerball dryer at a temperature of about 40-45° C. for a period of about 60 minutes. Hair on a separate mannequin head having the same level of curly hair was treated identically with a control of water only.

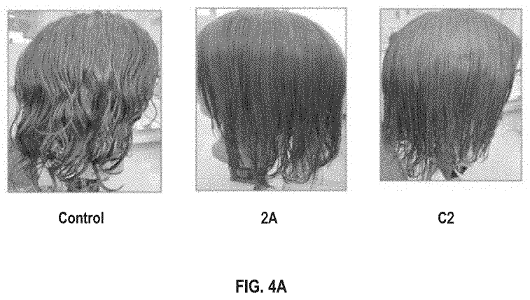

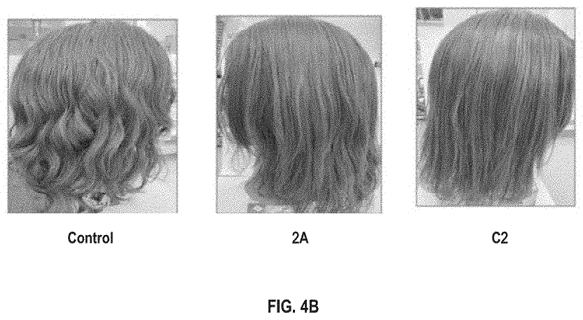

FIGS. 4A and 4B show the hair after either composition 2A or C2, or the control, is applied and while the hair is wet (FIG. 4A), and then once the hair is dried and the film is broken (FIG. 4B). As seen in the figures, the hair treated with compositions 2A and C2 both provide temporary straightening benefits to curly hair, as can be seen compared to the control. In addition, the hair treated with composition 2A demonstrated ease of application and detangling benefits over the hair treated with either the control or composition C2.

### Example 4C—Testing on Hair

Curly hair of 12 volunteers was washed and conditioned with standard shampoo and conditioner, and after excess water removed one half of the head of hair was treated with composition 1B and the other half of the head of hair was treated with composition C1, by spraying the compositions onto the hair followed by combing to straighten the hair. The hair was then dried with a rollerball dryer at a temperature of about 40-45° C. for a period of about 60 minutes. Once the hair was dry, the hair was combed with the fingers to break the film that had formed on the hair after drying.

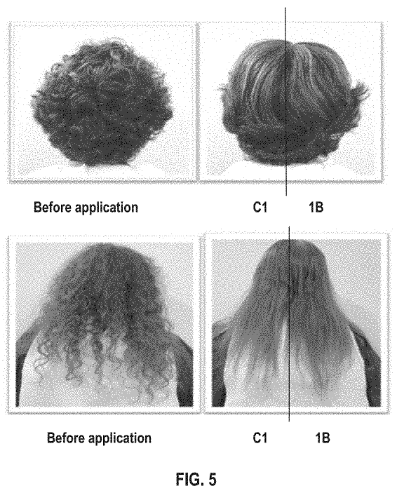

FIG. 5 shows the hair of 2 volunteers before treatment, and after the film was broken. As can be seen, the hair treated with compositions 1B and C1 both provide temporary straightening benefits to curly hair. Additionally, the hair treated with composition 1B had additional benefits such as volume reduction and frizz control.

The above Examples demonstrate that compositions according to the disclosure provide effective temporary straightening benefits to curly hair, while at the same time providing sensorial benefits such as smoothness and frizz-control to the hair. Further, compositions according to the disclosure have improved stability compared to compositions not according to the disclosure.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-32. (canceled)

33. A hair styling composition comprising:
(a) at least one polyphenol;
(b) at least one film former chosen from carboxylic acid polymers, polysaccharides, or mixtures thereof;
(c) at least one gum;
(d) at least one emulsifier;
(e) at least one fatty alcohol;
(f) at least one solvent comprising water; and
(g) optionally, at least one humectant.

34. The hair styling composition of claim 33, wherein the total amount of polyphenols ranges from about 0.01% to about 10% by weight, relative to the total weight of the composition.

35. The hair styling composition of claim 33, wherein the at least one polyphenol is chosen from flavonoid compounds.

36. The hair styling composition of claim 33, wherein the at least one polyphenol is chosen from tannic acid.

37. The hair styling composition of claim 33, wherein the total amount of film formers chosen from carboxylic acid polymers and polysaccharides ranges from about 0.01% to about 5% by weight, relative to the total weight of the composition.

38. The hair styling composition of claim 33, wherein the at least one film former comprises carrageenan.

39. The hair styling composition of claim 33, wherein the at least one gum is present in an amount ranging from about 0.01% to about 5% by weight, relative to the total weight of the composition.

40. The hair styling composition of claim 33, comprising:
(b) a film former comprising carrageenan; and
(c) at least one gum chosen from acacia, agar, algin, alginic acid, ammonium alginate, amylopectin, calcium alginate, carboxymethyl hydroxyethylcellulose, carnitine, cellulose acetate propionate carboxylate, cellulose gum, dextrin, diutan gum, gelatin, gellan gum, guar gum, hydroxyethylcellulose, hydroxypropylcellulose, hydroxypropyl methylcellulose, hydroxypropyl chitosan, hydroxypropyl guar, karaya gum, kelp, locust bean gum, methyl hydroxyethylcellulose, microcrystalline cellulose, natto gum, pectin, potassium alginate, propylene glycol alginate, sclerotium gum, sodium carboxymethyl dextran, sodium cellulose sulfate, tragacanth gum, xanthan gum, biosaccharide gum, or mixtures thereof.

41. The composition of claim 33, comprising:
(b) a film former comprising carrageenan; and
(c) a first gum comprising xanthan gum and a second gum comprising microcrystalline cellulose gum, wherein the weight ratio of xanthan gum:microcrystalline cellulose gum ranges from about 1:5 to about 1:15.

42. The hair styling composition of claim 33, comprising (a) tannic acid, present in an amount ranging from about 0.1% to about 5%, and (b) carrageenan, present in an amount ranging from about 0.01% to about 5%, wherein all amounts are by weight, relative to the total weight of the composition.

43. The hair styling composition of claim 33, wherein the at least one gum comprises microcrystalline cellulose, cellulose gum, sclerotium gum, xanthan gum, diutan gum, or mixtures thereof.

44. The hair styling composition of claim 33, wherein the total amount of emulsifier ranges from about 0.01% to about 5% by weight, relative to the total weight of the composition.

45. The hair styling composition of claim 33, wherein the fatty alcohol is chosen from straight or branched chain, saturated or unsaturated fatty alcohols having from 8 to 30 carbon atoms, derivatives thereof, or mixtures thereof.

46. The hair styling composition of claim 33, wherein the fatty alcohol is present in an amount ranging from about 0.01% to about 10% by weight, relative to the total weight of the composition.

47. The hair styling composition of claim 33, comprising at least one humectant chosen from glycerin, propylene glycol, butylene glycol, pentylene glycol, hexylene glycol, dipropylene glycol, diethylene glycol, diglycerol, hyaluronic acid, sorbitol, xylitol, inulin, or mixtures thereof.

48. The hair styling composition of claim 33, wherein the solvent comprises:
(a) at least 50% water; and
(b) optionally, at least one non-aqueous solvent,
wherein if the composition comprises at least one non-aqueous solvent, the total amount of non-aqueous solvent is less than about 20%, and
wherein all amounts are by weight, relative to the total weight of the composition.

49. A hair styling composition comprising:
(a) tannic acid;
(b) carrageenan;
(c) at least one gum chosen from microcrystalline cellulose, cellulose gum, sclerotium gum, xanthan gum, diutan gum, or mixtures thereof;
(d) at least one emulsifier;
(e) at least one fatty alcohol; and
(f) water, present in an amount of at least about 50% by weight, relative to the total weight of the composition.

50. The hair styling composition of claim 49, comprising:
(a) tannic acid, present in an amount ranging from about 0.5% to about 6%;
(b) carrageenan, present in an amount ranging from about 0.1% to about 1%;
(c) at least one gum chosen from microcrystalline cellulose, cellulose gum, sclerotium gum, xanthan gum, diutan gum, or mixtures thereof, present in an amount ranging from about 0.1% to about 2%;
(d) at least one emulsifier;
(e) at least one fatty alcohol, present in an amount ranging from about 1% to about 5%; and
(f) water, present in an amount of at least about 60%,
wherein all amounts are by weight, are relative to the total weight of the hair styling composition.

51. A method for styling hair comprising applying to the hair a hair styling composition comprising:
(a) at least one polyphenol;
(b) at least one film former chosen from carboxylic acid polymers, polysaccharides, or mixtures thereof;
(c) at least one gum;
(d) at least one emulsifier;
(e) at least one fatty alcohol;
(f) at least one solvent comprising water; and
(g) optionally, at least one humectant.

52. The method for styling hair according to claim 51, wherein the composition applied to the hair does not comprise a reducing agent, and no step of heating the hair occurs after the composition is applied to the hair.
